(19) United States
(12) Patent Application Publication
(10) Pub. No.: US 2003/0233097 A1
(43) Pub. Date: Dec. 18, 2003

(54) Artificial disc replacement (ADR) distraction sleeves and cutting guides

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/421,436

(22) Filed: Apr. 23, 2003

Related U.S. Application Data
(60) Provisional application No. 60/374,747, filed on Apr. 23, 2002.

(51) Int. Cl. A61F 5/00

(52) U.S. Cl. 606/86

(57) ABSTRACT

Apparatus and methods are effective in protecting the endplate of a vertebral body during use of a distraction tool. The preferred embodiment includes a flattened member having a first side configured for placement against an endplate of a vertebral body and a second side facing toward an intradiscal space, such that the insertion of a distraction tool into the intradiscal space will slide against the second side of the member as opposed to the vertebral body, thereby protecting the endplate. The flattened member may further include an anterior lip, and a guide formed through the lip to receive a cutting tool such as an osteotome or oscillating saw for resecting the vertebral body. The flattened member may also optionally include a posterior lip to prevent the cutting tool from extending too far past the vertebral body. As a different option, a t-shaped aperture may be formed through the lip to resect the vertebral body and cut a slot in the body to receive the keel of an artificial disc replacement (ADR).

### REFERENCE TO RELATED APPLICATION

This application claims priority from U.S. Provisional Patent Application Serial No. 60/374,747, filed Apr. 23, 2002, the entire content of which is incorporated herein by reference.

### FIELD OF THE INVENTION

This invention relates generally to artificial disc replacements (ADRs) and total disc replacements (TDRs) and, in particular, distraction sleeves useful in protecting vertebral endplates, certain of the sleeves including other useful features such as cutting guides.

### BACKGROUND OF THE INVENTION

Many spinal conditions, including degenerative disc disease, can be treated by spinal fusion or artificial disc replacement (ADR). ADR has several advantages over spinal fusion. The most important advantage of ADR is the preservation of spinal motion. Spinal fusion eliminates motion across the fused segments of the spine. Consequently, the discs adjacent to the fused level are subjected to increased stress. The increased stress increases the changes of future surgery to treat the degeneration of the discs adjacent to the fusion. However, motion through an ADR also allows motion through the facet joints. Motion across arthritic facet joints could lead to pain following ADR. Some surgeons believe patients with degenerative disease and arthritis of the facet joints are not candidates for ADR.

Current ADR designs do not attempt to limit the pressure across the facet joints or facet joint motion. Indeed, prior art ADR generally do not restrict motion. For example, some ADR designs place bags of hydrogel into the disc space. Hydrogel bags do not limit motion in any direction. In fact, bags filled with hydrogels may not provide distraction across the disc space. ADR designs with metal plates and polyethylene spacers may restrict translation but they do not limit the other motions mentioned above. The articular surface of the poly spacer is generally convex in all directions. Some ADR designs limit motion translation by attaching the ADR halves at a hinge.

FIG. 1A is a lateral view of a prior-art artificial disc replacement (ADR). FIG. 1B is an anterior view of a prior-art ADR. FIG. 1C is a drawing which shows the prior-art ADR in flexion, and FIG. 1D is a drawing which shows the device in extension. Note that, due to impingement, left bending as permitted by the typical prior-art device, increases pressure on the left facet, whereas right bending increases pressure on the right facet. Rotation increases pressure on the right facet and the left facet, and vice versa.

### SUMMARY OF THE INVENTION

This invention broadly resides in apparatus and methods for protecting the endplate of a vertebral body during use of a distraction tool. The preferred embodiment includes a flattened member having a first side configured for placement against an endplate of a vertebral body and a second side facing toward an intradiscal space, such that the insertion of a distraction tool into the intradiscal space will slide against the second side of the member as opposed to the vertebral body, thereby protecting the endplate.

The flattened member may further include an anterior lip, and a guide formed through the lip to receive a cutting tool such as an osteotome or oscillating saw for resecting the vertebral body. The flattened member may also optionally include a posterior lip to prevent the cutting tool from extending too far past the vertebral body. As a different option, a t-shaped aperture may be formed through the lip to resect the vertebral body and cut a slot in the body to receive the keel of an artificial disc replacement (ADR).

Typical use will involve two flattened members, one to protect each of the endplates of opposing vertebral bodies. The two flattened members may hinged, anteriorly, for example, to control distraction spacing.

### BRIEF DESCRIPTION OF THE DRAWINGS

FIG. 1A is a lateral view of a prior art artificial disc replacement (ADR);

FIG. 1B is an anterior view of a prior-art ADR;

FIG. 1C is a drawing which shows a prior-art ADR in flexion;

FIG. 1D is a drawing which shows the device in extension;

FIG. 2 is a simplified drawing of a restricted motion ADR according to the present invention;

FIG. 3A is a drawing of the embodiment of FIG. 2 in flexion;

FIG. 3B is a drawing of the embodiment of FIG. 2 in extension;

FIG. 3C is an anterior view of the embodiment of FIG. 2 attached to adjacent vertebrae;

FIG. 3D is a drawing of the embodiment of FIG. 2 illustrating how lateral bending is limited by contact on the left when bending is to the left, and on the right when bending is to the right;

FIG. 3E is a lateral view of a restricted motion ADR according to the invention;

FIG. 4 is a drawing of an alternative embodiment of the invention;

FIG. 5 is a side-view drawing which illustrates a way in which screws may be used to fix an ADR;

FIG. 6 is a drawing which shows the use of anterior flanges;

FIG. 7A is a side-view drawing of a further alternative embodiment according to the invention;

FIG. 7B shows the flange device of FIG. 7A in flexion;

FIG. 8 is a side-view drawing showing the use of an anterior check rein to prevent extension, for example;

FIG. 9 depicts the use of cross-coupled check reins;

FIG. 10 illustrates the optional use of an anterior flange configured to inhibit extension;

FIG. 11A is a drawing which illustrates yet a different embodiment of the invention;

FIG. 11B is a drawing which shows the device of FIG. 11A in flexion;

FIG. 11C shows the device in extension;

FIG. 11D is a side-view drawing of the way in which screws may be used to hold the device of FIG. 11D in place;

FIG. 11E an A-P view;

FIG. 12 is a side-view drawing which shows the area that could be removed to customize the vertebrae;

FIG. 13 is a first version according to this embodiment illustrating rotation surface(s);

FIG. 14 is a side-view drawing which shows a partial rotation surface received by a concavity in the imposing endplate;

FIG. 15A is an end-view of an ADR according to the invention placed on the vertebrae seen from a top-down A-P view;

FIG. 15B is a drawing of the embodiment of FIG. 15A with the ADR and axle rotated;

FIG. 16 is a drawing which shows a removable alignment guide used for placement of this embodiment;

FIG. 17 is a simplified cross-sectional view of a patient on an operating table, showing the alignment guide in position;

FIG. 18A is a lateral view using fluoroscopy which shows the circular cross-section of the axle when properly aligned;

FIG. 18B is an anterior view of this alternative embodiment;

FIG. 18C is an anterior view;

FIG. 19A shows how disc space is distracted;

FIG. 19B shows the impact distraction element in place between the end plates;

FIG. 19C shows the tool being manipulated to spread the vertebrae apart;

FIG. 19D shows a third step how the end plates are prepared through the use of a reamer and/or circular grinder;

FIG. 19E shows a first end plate for the final ADR is inserted;

FIG. 19F shows how the second end plate is inserted;

FIG. 19G show how the end plates are optionally screwed into place;

FIG. 19H shows the step of inserting an axle between the end plates;

FIG. 19I shows the anterior poly block snapped in position on the other side of the installed axle;

FIG. 20 is an anterior view of the ADR installed between opposing vertebrae;

FIG. 21 shows the use of optional wedges or convex pieces to attach the ADR end plate;

FIG. 22 is a drawing which shows an inventive cutting guide having a curved end to prevent saw from cutting into the nerves;

FIG. 23A is a side-view drawing of a further, different embodiment of the invention utilizing a hinged axle;

FIG. 23B is an end view of the embodiment of FIG. 23A shown without flexion/extension blocks to better illustrate the hinged portion;

FIG. 24A is a lateral view of an alternative embodiment of distraction sleeves according to the invention;

FIG. 24B is an anterior view of the embodiment of the distraction sleeves drawn in FIG. 24A;

FIG. 24C is an anterior view of yet a further alternative embodiment of the distraction sleeves;

FIG. 24D is a view of the lateral aspect of a distractor according to the invention, which is impacted between the distraction sleeves;

FIG. 24E is a view of the lateral aspect of an alternative distractor of the embodiment of the device drawn in FIG. 24D;

FIG. 24F is a view of the lateral aspect of the spine, the distraction sleeves, an impacted distraction tool (dotted area of the drawing), and a sawblade or osteotome;

FIG. 25A shows the side of an alternative embodiment of a distractor according to the invention; and

FIG. 25B is a view of the front of the embodiment of the distractor drawn in FIG. 25A.

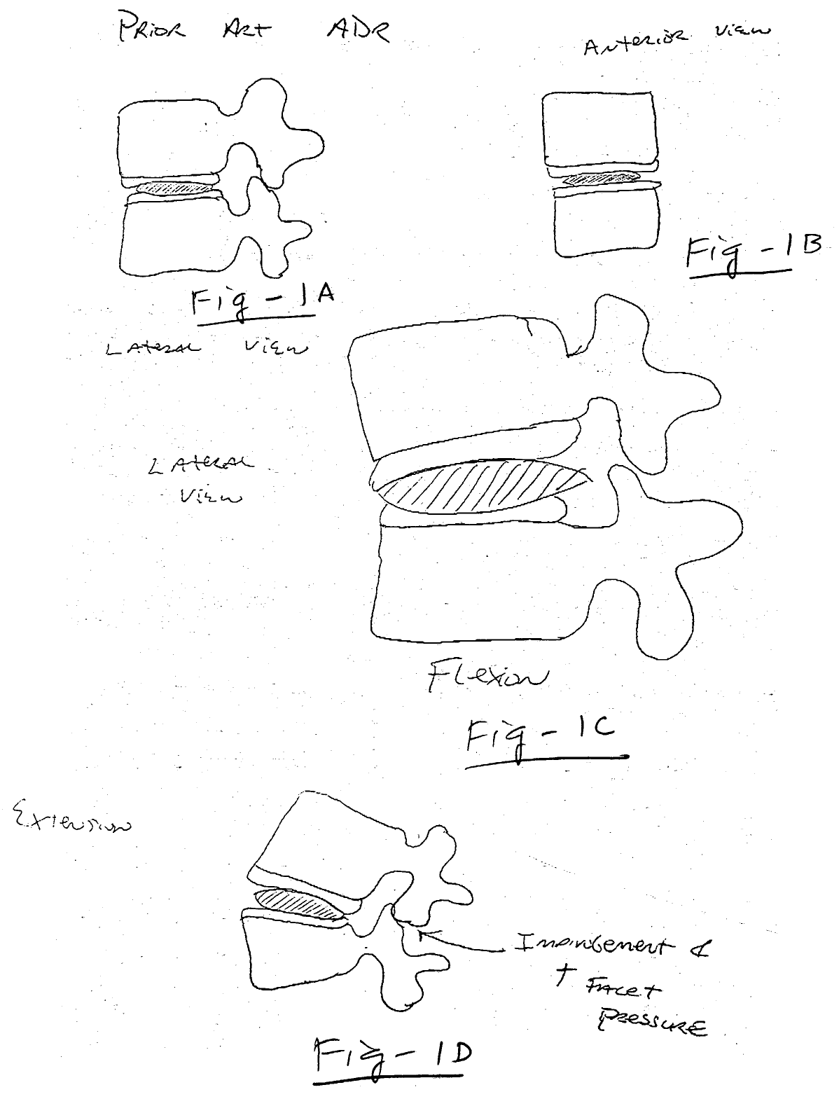

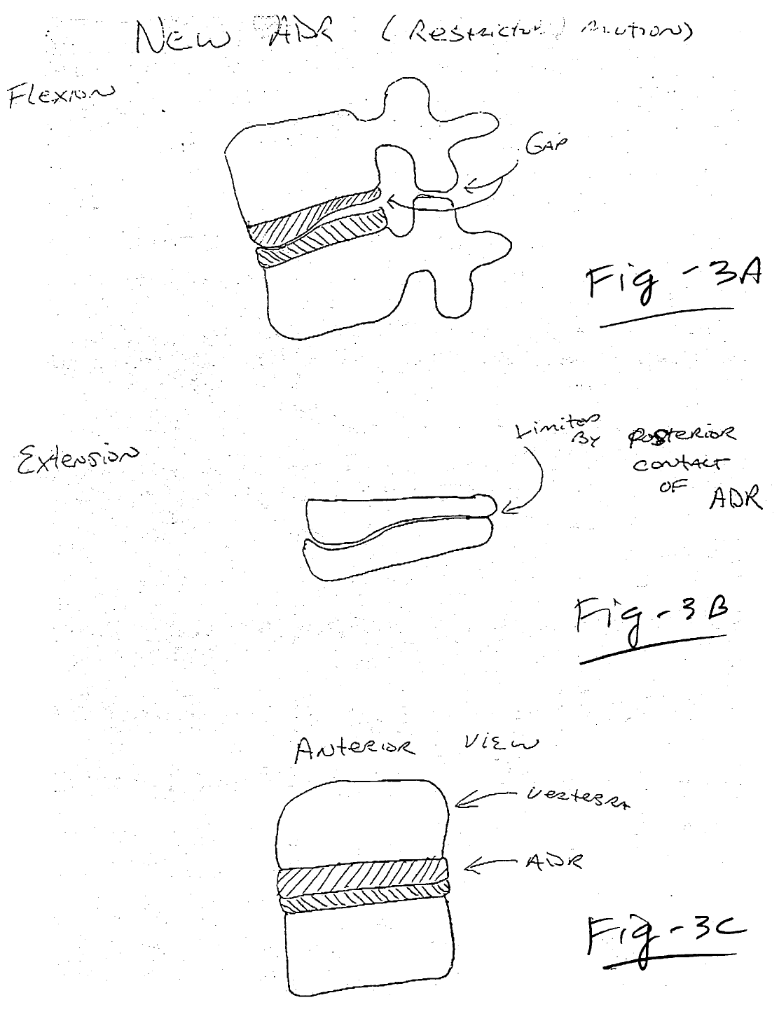

### DETAILED DESCRIPTION OF THE INVENTION

The present invention limits both facet joint pressure and facet joint motion. Broadly, the pressure on the facet joints is lowered from the preoperative pressure by distracting the disc space. The present invention also reduces the facet joint pressure by eliminating or significantly reducing motion across the ADR that increase the pressure on the facet joints. Specifically, ADR design in accordance with the various embodiments restricts spinal extension, rotation, translation, and lateral bending. Forward flexion is not restricted as forward flexion decreases the pressure on the facet joints.

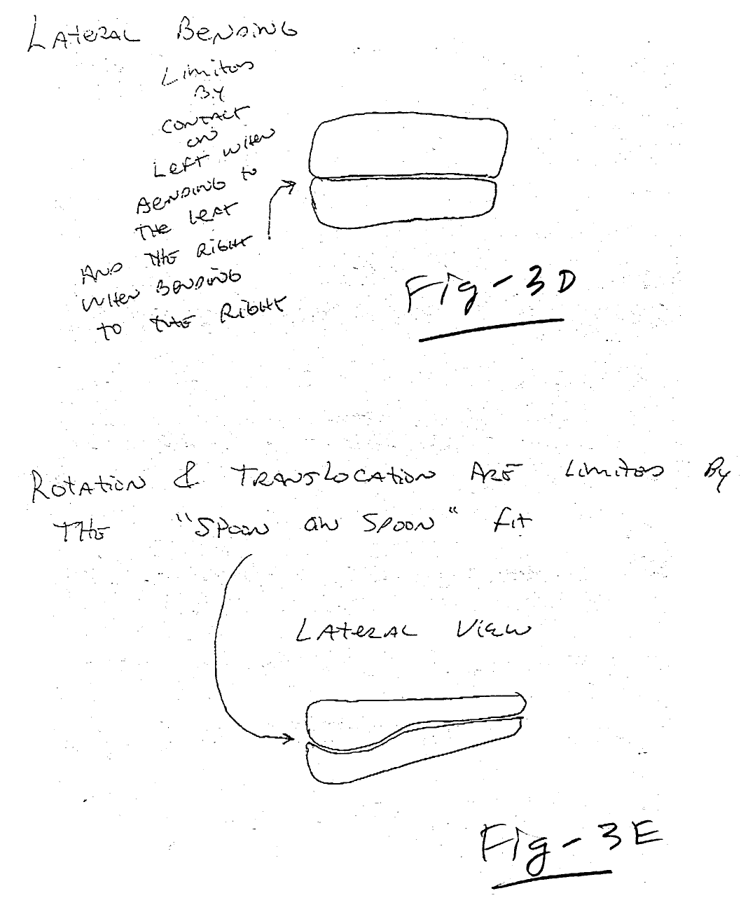

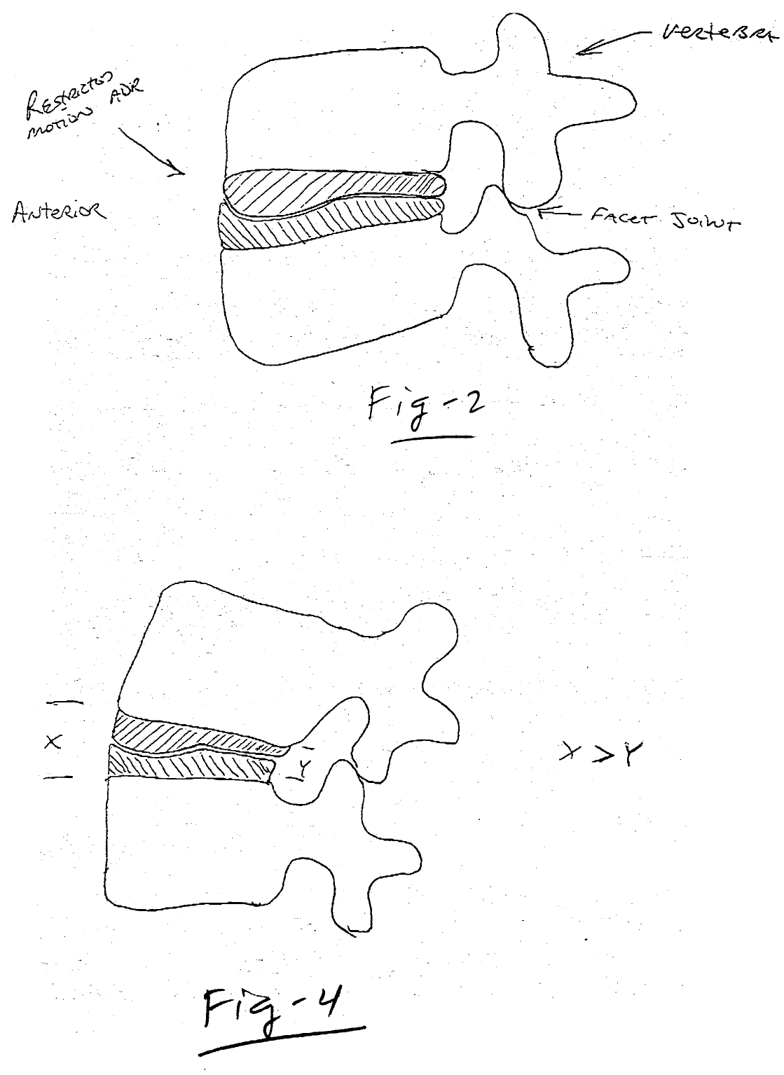

FIG. 2 is a simplified drawing of a restricted motion artificial disc replacement (ADR) according to this invention. FIG. 3A is a drawing of the embodiment of FIG. 2 in flexion, illustrating the way in which gaps are created in the posterior of the vertebrae and the facet joint. FIG. 3B is a drawing of the embodiment of FIG. 2 in extension, showing how posterior contact is limited. FIG. 3C is an anterior view of the embodiment of FIG. 2 attached to adjacent vertebrae. FIG. 3D is a drawing of the embodiment of FIG. 2 illustrating how lateral bending is limited by contact on the left when bending is to the left, and on the right when bending is to the right. FIG. 3E is a lateral view of a restricted motion ADR according to the invention, illustrating how rotation and translocation are limited by a spoon-on-spoon cooperation.

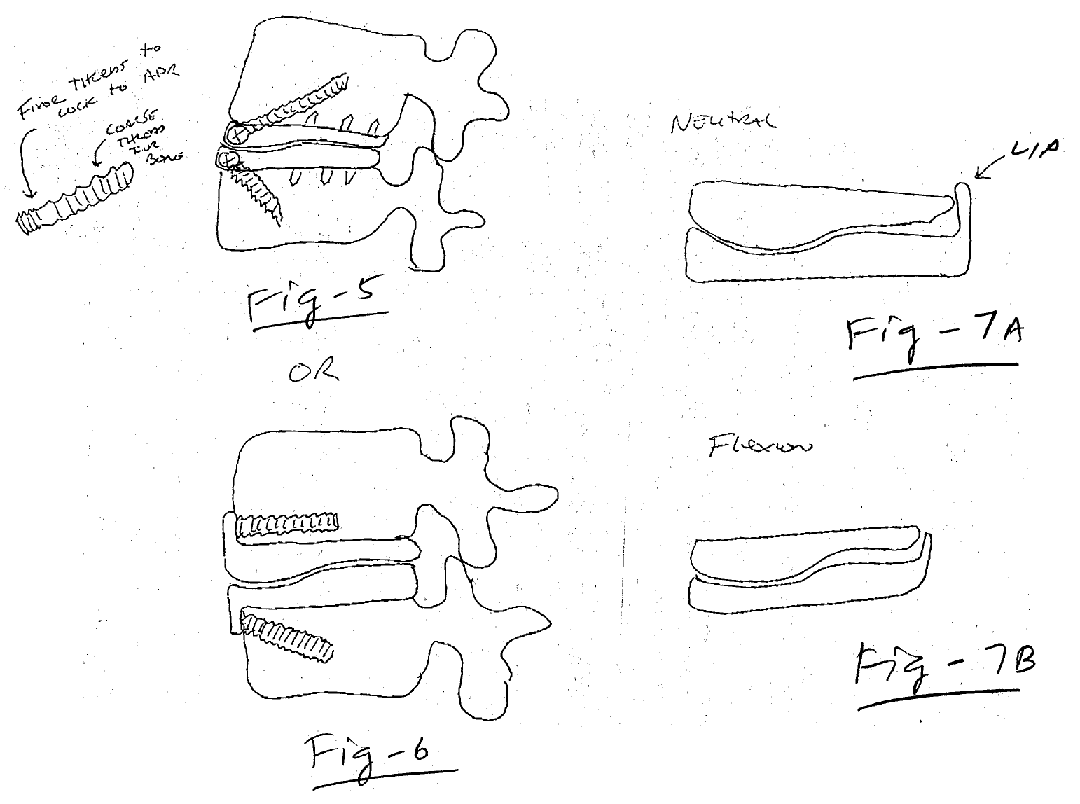

FIG. 4 is a drawing of an alternative embodiment of the invention, illustrating how a wedge or trapezoid-shaped ADR may be used according to the invention to preserve lordosis. FIG. 5 is a side-view drawing which illustrates a way in which screws may be used to fix an ADR according to the invention to upper and lower vertebrae. In particular, a fastener may be used having coarse threads received by the bone, and finer threads associated with actually locking the ADR into place. FIG. 6 is a drawing which shows the use of anterior flanges facilitating the use of generally transverse as opposed to diagonally oriented screws.

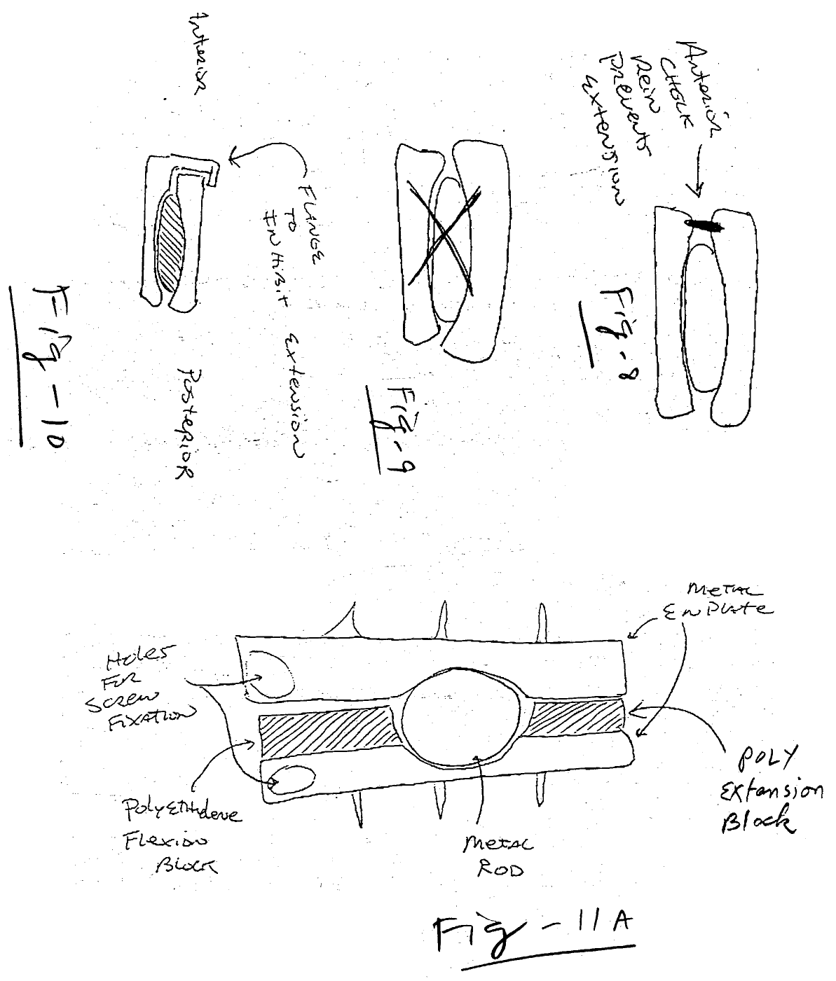

FIG. 7A is a side-view drawing of a further alternative embodiment according to the invention, featuring an optional lip to prevent the trapping of soft tissue during the movement from a flexion to neutral position. FIG. 7B shows the flange device of FIG. 7A in flexion. As a substitute for, or in conjunction with, peripheral flanges, check reins may be used to restrict motion. FIG. 8 is a side-view drawing showing the use of an anterior check rein to prevent extension, for example. Lateral check reins may be used to prevent lateral bending, and cross-coupled check reins may be used to prevent translation. FIG. 9 depicts the use of cross-coupled check reins. FIG. 10 illustrates the optional use of an anterior flange configured to inhibit extension.

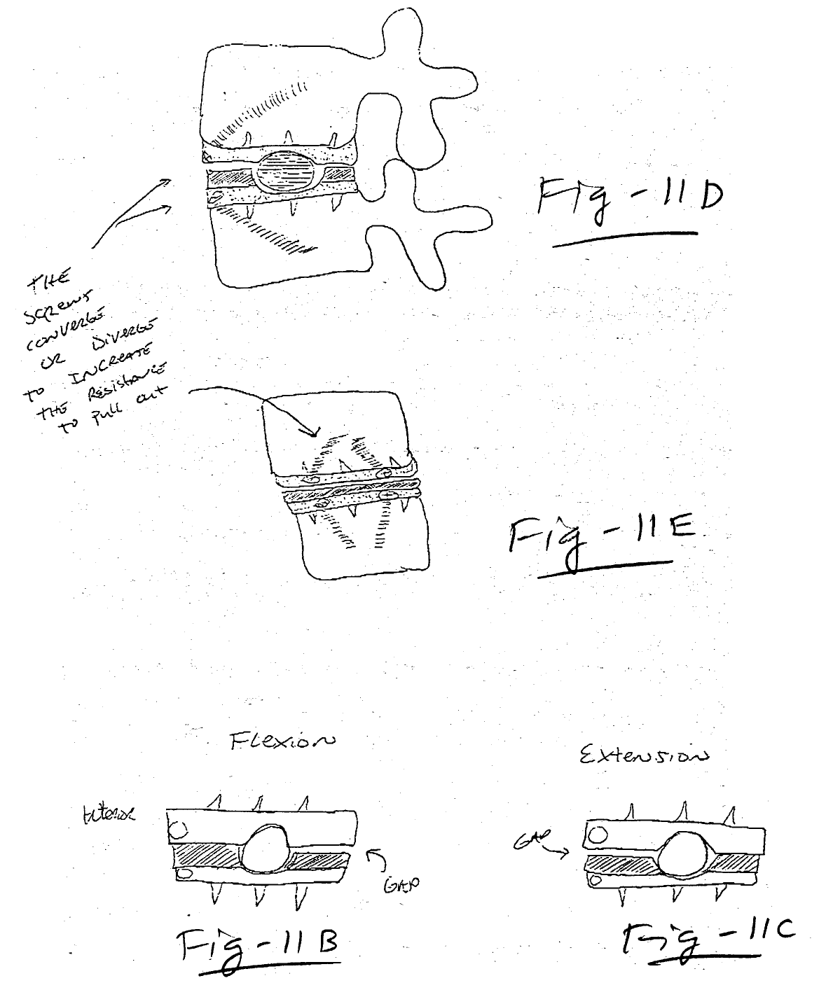

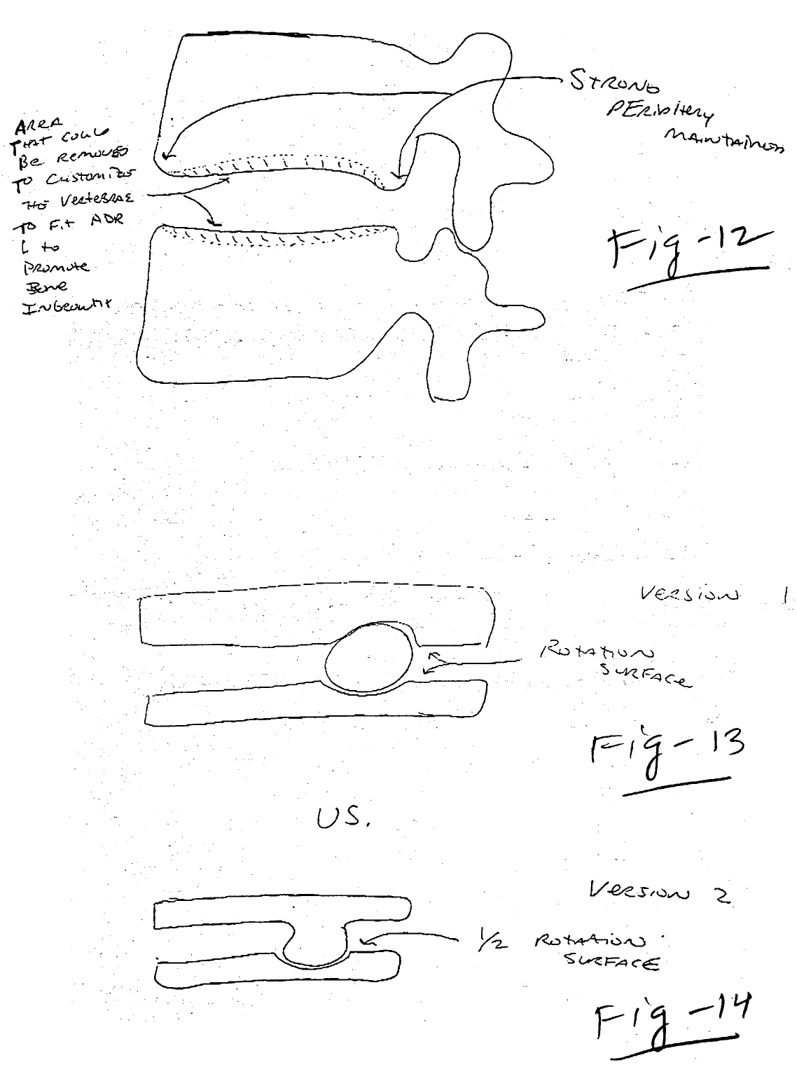

FIG. 11A is a drawing which illustrates yet a different embodiment of the invention, including the use of flexion and/or extension blocks. Shown in the figure, endplates, preferably metal, include recesses to receive a centralized rod, also preferably metallic. On either side of the rod, but between the end plates, there is disposed a more wearing bearing block of material such as polyethylene, one preferably associated with flexion and an opposing block associated with extension. Holes may be provided for fixation along with projections for enhanced adherence. FIG. 11B is a drawing which shows the device of FIG. 11A in flexion, and FIG. 11C shows the device in extension. Note that, during flexion, a posterior gap is created, whereas, in extension, an anterior gap is created. In this embodiment, the degree of flexion and extension may be determined by the thickness of the flexion/extension blocks, which may determined at the time of surgery. FIG. 11D is a side-view drawing of the way in which screws may be used to hold the device of FIG. 11D in place. FIG. 11E an A-P view. Note that the screws may converge or diverge, to increase resistance to pull-out.

The superior surface of the superior endplate and the inferior surface of the inferior endplate of the ADR could be convex. The convex surfaces of the ADR would fit the concavities of the endplates of the vertebrae. The endplates could be decorticated to promote bone ingrowth into the endplates of the ADR. An expandable reamer or a convex reamer could preserve or increase the concavities. The concavities have two important advantages. First, they help prevent migration of the ADR. The convexities of the ADR fit into the concavities of the vertebrae. Second, the majority of support for the ADR occurs at the periphery of the vertebral endplates. Thus, reaming away a portion of the central, concave, portion of the vertebrae promotes bone ingrowth through exposure to the cancellous interior of the vertebrae, yet preserves the stronger periphery. FIG. 12 is a side-view drawing which shows the area that could be removed to customize the vertebrae so as to fit an ADR according to the invention and/or promote ingrowth.

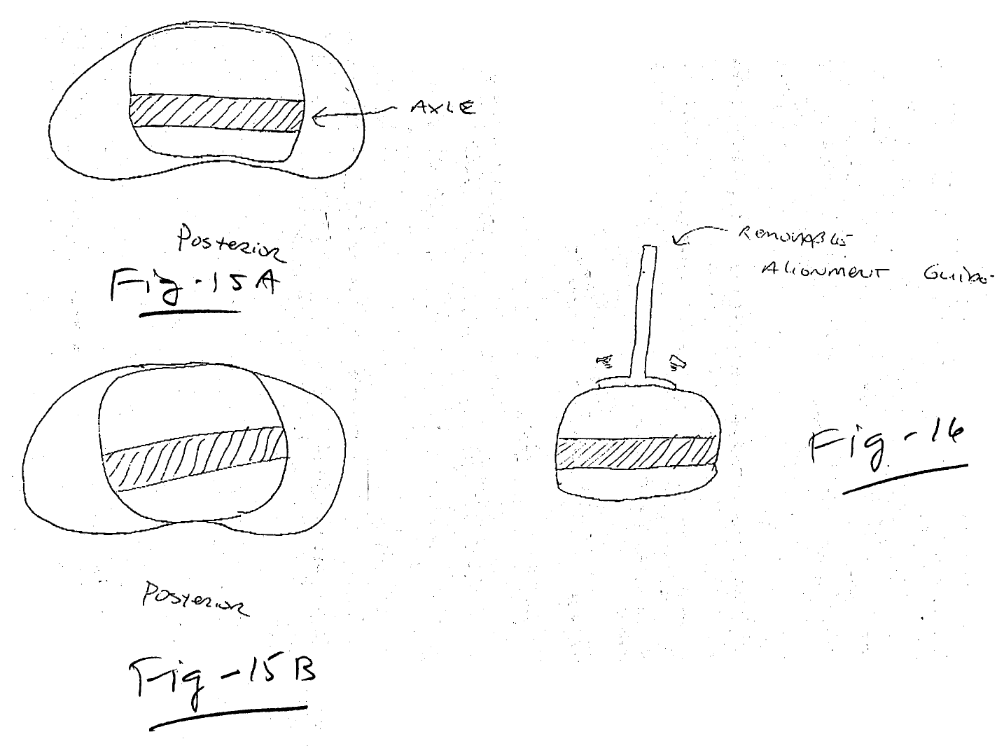

The endplates of the ADR could be any material that promotes bone ingrowth. For example, titanium or chrome-cobalt with a porous, beaded, or plasma spray surface. The flexion and extension blocks would likely be made of polyethylene, but could also be made of other polymers, ceramic, or metal. The central rod or axle would likely made of the same metal as the endplates of the ADR, but could also be made of polyethylene or other polymer, or ceramic. A metal or ceramic rod would have better surface wear than a polyethylene rod. A limited amount of compression to axial loads could occur when a portion of the ADR endplates lie against the polyethylene blocks. A central rod is preferred over incorporating a raised rod like projection into one of the endplates. The central rod allows rotation about twice as much surface area (the superior and inferior surfaces). The increased surface area decreases the pressure on the surface during rotation about the central axle/rod. FIG. 13 is a first version according to this embodiment illustrating rotation surface(s). FIG. 14 is a side-view drawing which shows a partial rotation surface received by a concavity in the imposing endplate. Both versions shown in FIGS. 13 and 14 are assembled within the disc space.

Alignment of the ADR is critical. If the central rod or axle is diagonal to the long axis of the vertebral endplate, the patient will bend to the left or right while bending forward. Novel (for and ADR) alignment guides are described below. Furthermore, if the axle is made of polyethylene, metallic markers will be incorporated into the ends of the axle. Surgeons can assure proper alignment by fluoroscopic images during surgery. FIG. 15A is a end-view of an ADR according to the invention placed on the vertebrae seen from a top-down A-P view. FIG. 15B is a drawing of the embodiment of FIG. 15A with the ADR and axle rotated. Should the patient have trouble bending forward, and so forth, the patient may twist at the side while bending forward, as appropriate.

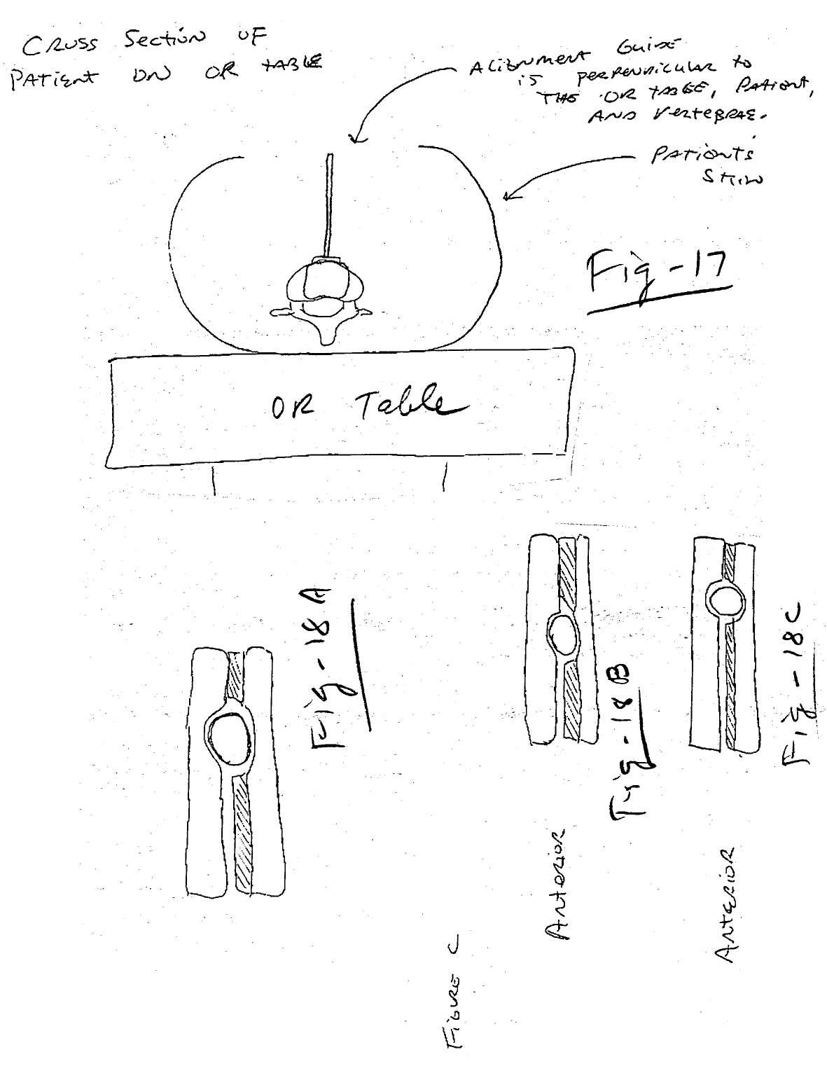

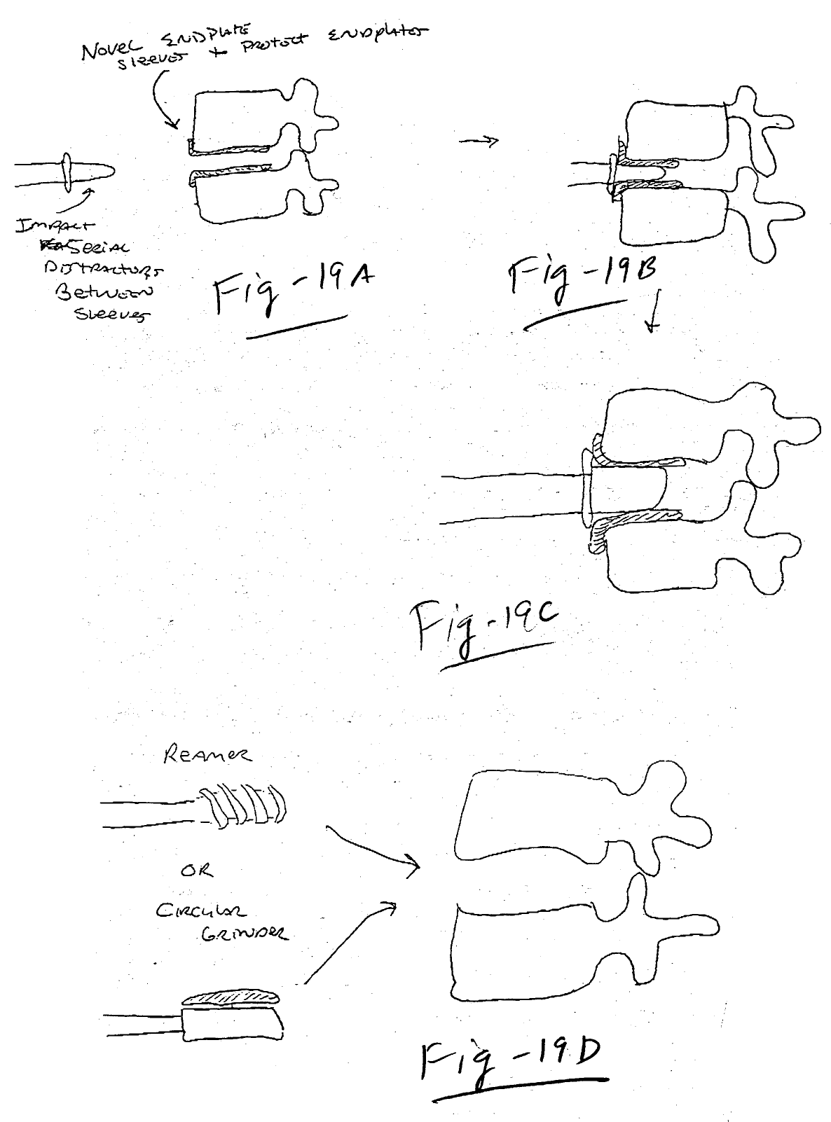

FIG. 16 is a drawing which shows a removable alignment guide used for placement of this embodiment. FIG. 17 is a simplified cross-sectional view of a patient on an operating table, showing the alignment guide in position. In particular, the alignment guide is preferably perpendicular to the table, the patient, and vertebrae with respect to al proper orientation. FIG. 18A is a lateral view using fluoroscopy which shows the circular cross-section of the axle when properly aligned.

The ADR endplates could be designed to locate the axle transversely in any location from anterior to posterior. The location may vary depending on the disc that will be replaced. For example, the axle may located at the junction of the anterior ⅔ rd and posterior ⅓^{rd} for the L5/S1 disc but at the anterior ½ and posterior ½ for the L3/L4 disc. Similarly, the degree of wedge shape will vary with the disc to be replaced. L5/S1 will require a more wedge shaped ADR than L3/L4. FIG. 18B is an anterior view of this alternative embodiment, and FIG. 18C is an anterior view.

Preoperative templates will be provided to help the surgeon predict which ADR will be needed. The ADR could be inserted fully assembled or constructed in the disc space. Construction within the disc space allows the surgeon to force spikes of the ADR endplate into the vertebrae. Assembly in the disc space also allows maximum use of the vertebral concavities. The polyethylene blocks contain features to allow them to snap into place. Polyethylene trays with “snap” features are well described in the total knee replacement literature.

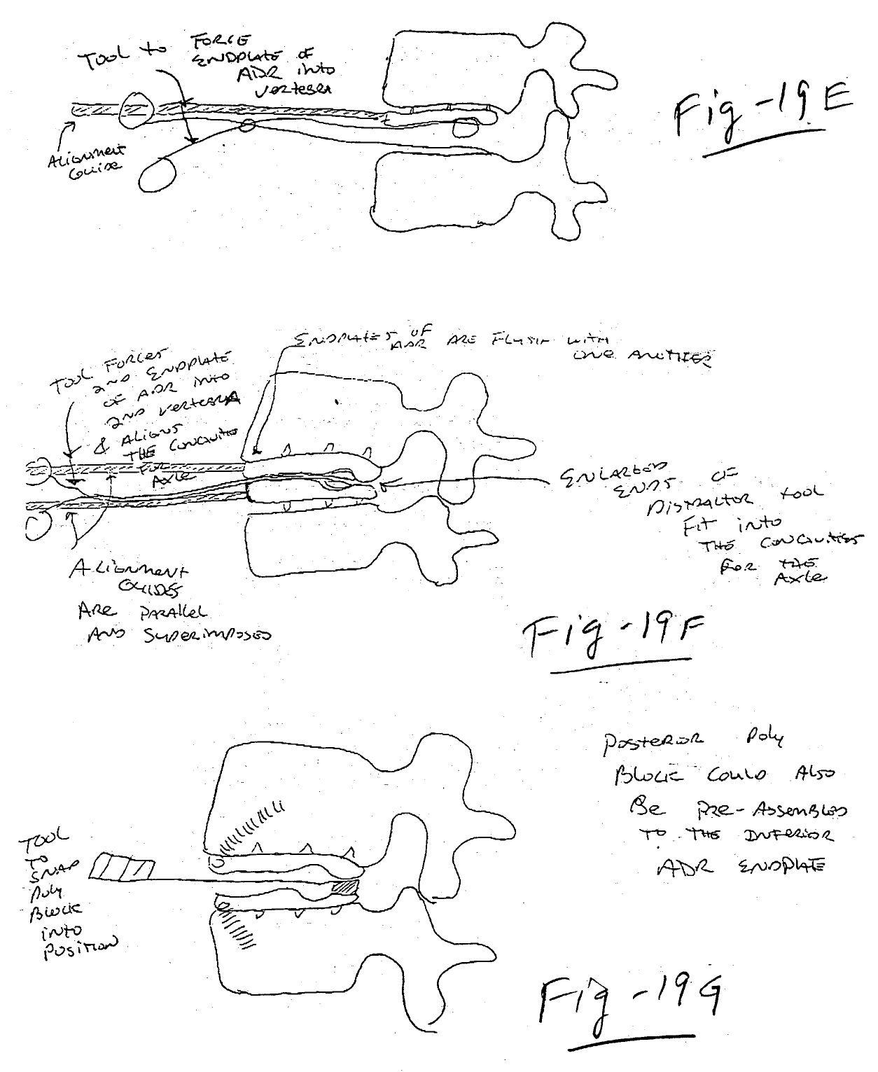

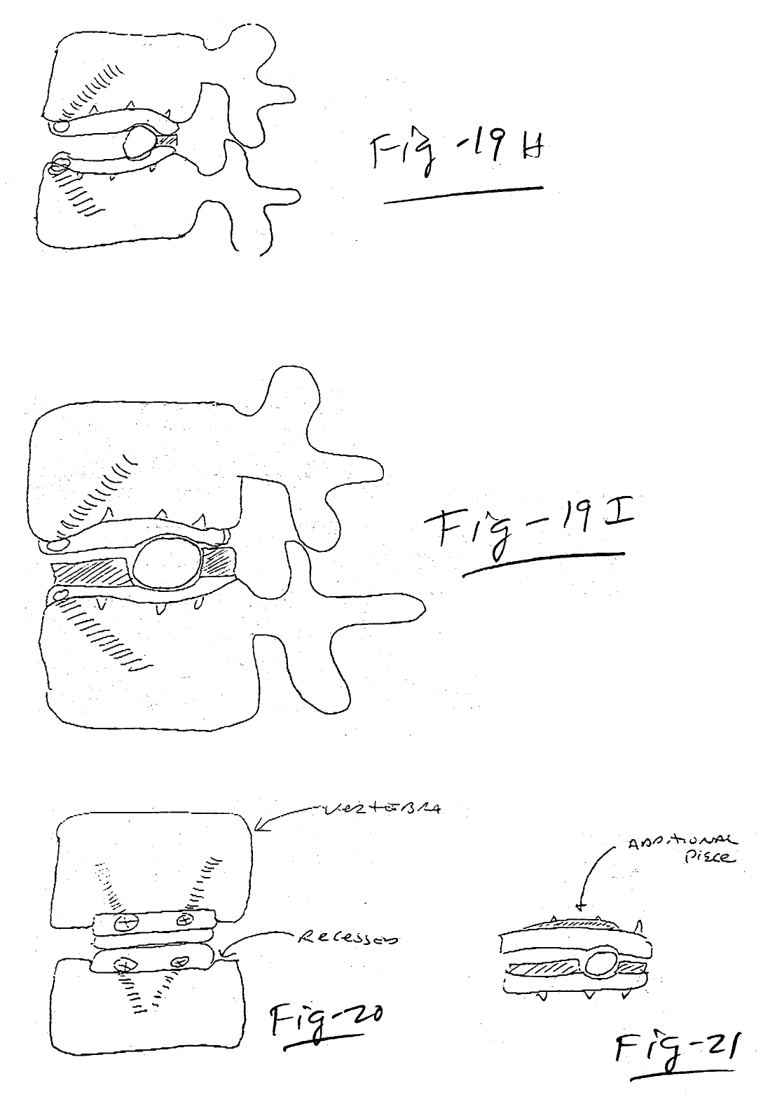

FIGS. 19A-19I illustrate steps associated with installing a restricted motion ADR according to the invention. In the preferred embodiment the ADR relies on bone ingrowth. Alternatively, the ADR may be cemented to the vertebrae using, for example, methyl methacrylate. Novel, safer cutting guides, and a novel distraction instruments are described. The system also provides trial implants and instruments to determine the balance and tension of the surrounding soft tissues.

As an initial step, a portion of the disc annulus and most or all of the disc nucleus are removed (not shown). As a second step, the disc space is distracted, as shown in FIG. 19A. In this case a novel implant sleeve is used to protect the end plates, and an impact serial distracter is used between these sleeves. FIG. 19B shows the impact distraction element in place between the end plates, and FIG. 19C shows the tool being manipulated to spread the vertebrae apart.

According to a third step, the end plates are prepared through the use of a reamer and/or circular grinder with the distraction sleeves removed, as shown in FIG. 19D. As a fourth step, the trial ADR is inserted (not shown) so as to select a proper size ADR (step 5, also not shown). Having determined the proper size, a first end plate for the final ADR is inserted as shown in FIG. 19E with a tool used to force the end plate of the ADR into the vertebrae, whether upper or lower.

This section of the disclosure emphasizes methods and instruments that allow for the separate insertion of ADR EPs. Aligning the insertion of a second ADR EP relative to a first EP that enables the use of longer projections from the ADR EPs, resulting in a more controlled procedure. Referring to FIGS. 19E and 19F in particular, the upper ADR EP has been press fit into the vertebra above the disc space. A special tool fits into a portion of the ADR EP that was inserted first, thereby aligning the insertion of the second ADR. The tool can also be used to press the second ADR EP into the vertebra. Although FIGS. 19E and 19F illustrate the use of an instrument that fits into cylinder-like concavities, the instrument could fit into other shapes in the ADR EPs, including slots and other shapes with flat sides.

In FIG. 19F, the second end plate is inserted, such that the opposing end plates are flush with one another. The tool used for this purpose forces the second plate of the ADR into the second vertebrae while simultaneously aligning the concavities to receive the axle. Alignment guides may be used in parallel/superimposed fashion to ensure that the opposing end plates are oriented properly. In addition, the enlarged ends of the distraction tool may include end features which fit into the cavities for axle, again, to ensure proper orientation. In step 8, shown in FIG. 19G, the end plates are optionally screwed into place, and a first poly block is installed posteriorly using a tool to snap the block into position. Note that the posterior poly block may also be preassembled to the inferior ADR end plate, as an option.

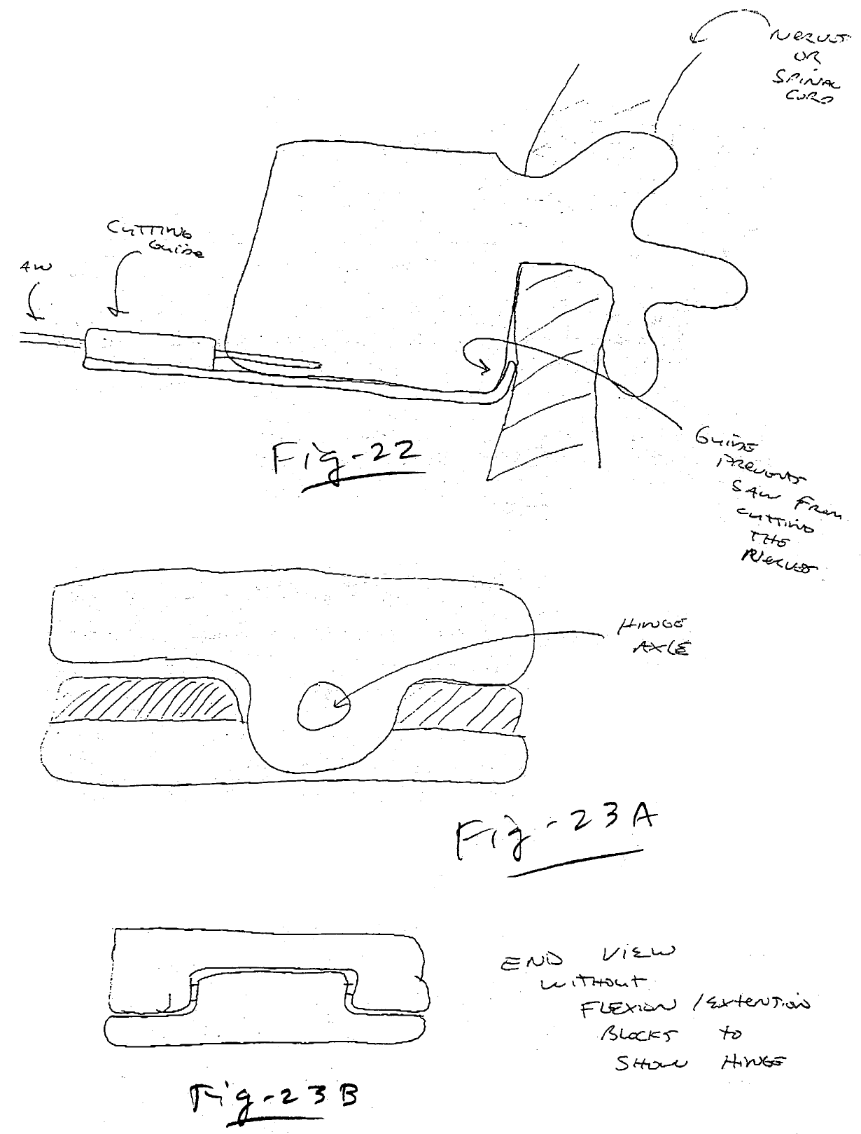

FIG. 19H shows the step of inserting an axle between the end plates. In step 10, shown in FIG. 19I, the anterior poly block is snapped in position on the other side of the installed axle. The ADR could be placed into recessed areas of the vertebrae to help hold it in place. FIG. 20 is an anterior view of the ADR installed between opposing vertebrae also showing the relative positioning of recesses formed in the end plates of the vertebrae. FIG. 21 shows the use of optional wedges or convex pieces to attach the ADR end plate so as to customize the prosthesis to a particular patient anatomy.

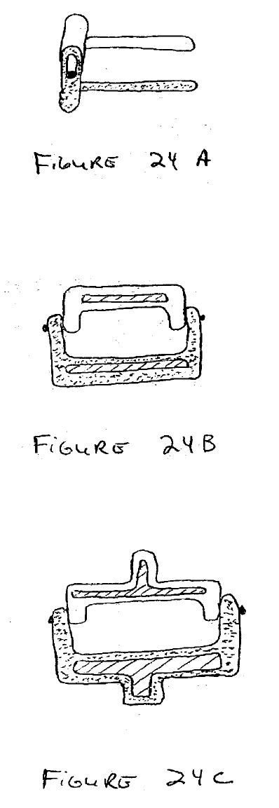

FIG. 22 is a drawing which shows an inventive cutting guide having a curved end to prevent saw from cutting into the nerves. FIG. 23A is a side-view drawing of a further, different embodiment of the invention utilizing a hinged axle. FIG. 23B is an end view of the embodiment of FIG. 23A shown without flexion/extension blocks to better illustrate the hinged portion.

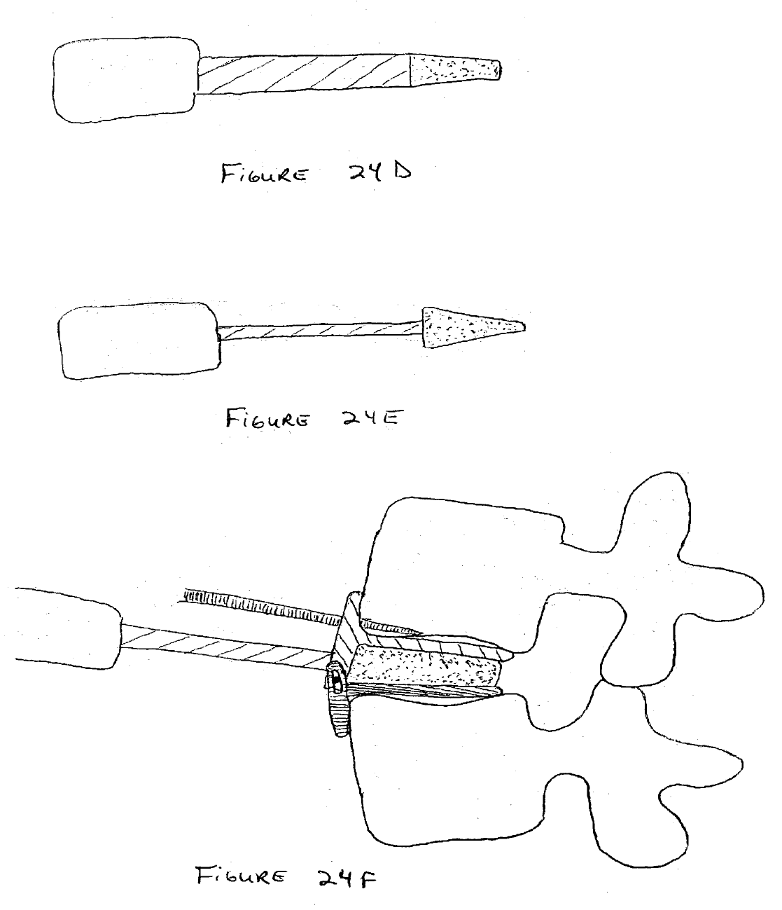

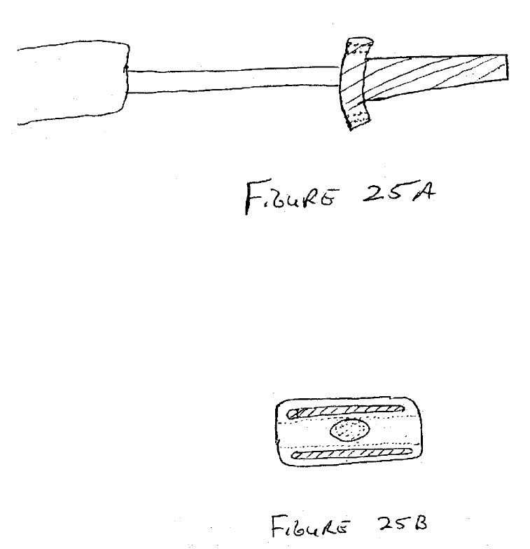

FIG. 24A is a lateral view of an alternative embodiment of distraction sleeves according to the invention. In this case the sleeves are hinged together, which permits separation of the distraction sleeves. The hinge joint also permits tilting of one sleeve relative to the other sleeve, which permits the introduction of a wedge-shaped instrument to separate the vertebrae.

FIG. 24B is an anterior view of the embodiment of the distraction sleeves drawn in FIG. 24A. Note that the sleeves may contain slots to cut the vertebrae. The slots are illustrated by the area of the drawing with diagonal lines. The distraction sleeves would preferably be made available in various sizes with the osteotomy slots or saw guides to remove a variable portion of the vertebral endplates. For example, the sleeves could be sized to remove 1-5 mm of the vertebrae, in 1 mm increments.

FIG. 24C is an anterior view of yet a further alternative embodiment of the distraction sleeves. The slots align tools to cut the ends of the vertebrae and to cut slots in the vertebrae to receive the keels of an ADR.

FIG. 24D is a view of the lateral aspect of a distractor according to the invention, which is impacted between the distraction sleeves. Such distractors would also come in various sizes and shapes. For example the tip of the distractor (dotted area of the drawing) could range for 3 mm to 20 mm in height, in 1 mm increments. Furthermore, the distractor tips could vary in the degrees of wedge shape. For example, the tip could have 0-20 degrees of wedge shape, in 1 degree increments.

FIG. 24E is a view of the lateral aspect of an alternative distractor of the embodiment of the device drawn in FIG. 24D. The tip of the distractor is more wedge-shaped than that drawn in FIG. 24D, and the shaft of the tool is smaller in diameter. The shafts of the distractors could vary in diameter. Shafts with smaller diameters would flex, thus preventing the application of excessive force between the distraction sleeves. Alternatively, one or preferably two instruments could be inserted between the distraction sleeves to cam open the disc space. Similarly, a scissor action distraction tool could be used between the distraction sleeves. Lastly, the distraction sleeves could have components that extend into the spinal canal as illustrated in FIG. 22.

FIG. 24F is a view of the lateral aspect of the spine, the distraction sleeves, an impacted distraction tool (dotted area of the drawing), and a sawblade or osteotome. The saw blade or osteotome (area of the drawing with vertical lines) can be seen projecting into the vertebra through the slot in the superior distraction sleeve.

FIG. 25A shows the side of an alternative embodiment of a distractor according to the invention. The one piece distractor has an intradiscal component and slots for cutting the endplates of the vertebrae. As described with reference to FIG. 24D, the distractor could be sized to remove a variable portion of the vertebrae. Furthermore, the distractor could be sized to provide a variable amount of distraction. Lastly, various shapes of the distractor would permit variable degrees of wedge shape to the distracted disc space. The dotted area of the drawing represents slots in the distractor for a sawblade or an osteotome.

FIG. 25B is a view of the front of the embodiment of the distractor drawn in FIG. 25A. The shaft of the distractor is seen in cross section (dot filled circle). The dotted lines represent the location of the intradiscal component. The intradiscal component is located on the posterior portion of the tool. The rectangles with diagonal lines represent the slots.

I claim:

1. Apparatus for protecting the endplate of a vertebral body during use of a distraction tool, comprising:
a flattened member having a first side configured for placement against an endplate of a vertebral body and a second side facing toward an intradiscal space,
such that the insertion of a distraction tool into the intradiscal space will slide against the second side of the member as opposed to the vertebral body, thereby protecting the endplate.

2. The apparatus of claim 1, wherein:
the flattened member includes an anterior lip; and
a guide formed through the lip to receive a cutting tool for resecting the vertebral body.

3. The apparatus of claim 1, wherein:
the flattened member includes an anterior lip with a guide to receive a cutting tool for resecting the vertebral body; and
a posterior lip to prevent the cutting tool from extending too far past the vertebral body.

4. The apparatus of claim 1, wherein:
the flattened member includes an anterior lip; and
a t-shaped aperture formed through the lip to resect the vertebral body and cut a slot in the body to receive the keel of an artificial disc replacement (ADR).

5. The apparatus of claim 1, including two flattened members, one to protect each of the endplates of opposing vertebral bodies.

6. The apparatus of claim 5, wherein the two flattened members are hinged.

7. The apparatus of claim 5, wherein the two flattened members include an anterior hinge.
